(19) United States
(12) Patent Application Publication
(10) Pub. No.: US 2023/0287170 A1
(43) Pub. Date: Sep. 14, 2023

(54) FLUORESCENT DYES COMPRISING M-CONJUGATED 1,1 -BINAPHTHYL-BASED POLYMERS

(71) Applicant: Miltenyi Biotec B.V. & Co. KG, Bergisch Gladbach (DE)

(72) Inventors: Christian Dose, Kürten (DE); Dirk Meineke, Köln (DE); Daniel Berndt, Lindlar (DE)

(73) Assignee: Miltenyi Biotec B.V. & Co. KG, Bergisch Gladbach (DE)

(21) Appl. No.: 18/015,351

(22) PCT Filed: Jul. 13, 2021
(86) PCT No.: PCT/EP2021/069420

§ 371 Date: Jan. 10, 2023

(30) Foreign Application Priority Data
Jul. 16, 2020 (EP) 20186344.6
Jul. 13, 2021 (WO) PCT/EP2021/069420

(51) Int. Cl. C08G 61/12; G01N 33/58

(52) U.S. Cl. CPC C08G 61/126; G01N 33/582; C08G 2261/1424; C08G 2261/122; C08G 2261/124; C08G 2261/1644; C08G 2261/18; C08G 2261/214; C08G 2261/314; C08G 2261/411; C08G 2261/522; C08G 2261/72; C08G 2261/94

(57) ABSTRACT

The invention is directed to a conjugate having the general formula (I) With AR, MU and L1 as repeating units of a polymer MU is a polymer modifying unit or band gap modifying unit that is evenly or randomly distributed along the polymer main chain, L1 is an aryl or a heteroaryl group evenly or randomly distributed along the polymer, L2 is an aryl or a heteroaryl group located on the ends of the polymer, FL is a fluorescent moiety, G1 and G2 stand for hydrogen, halogen or an antigen recognizing moiety, with the provision than at least one of G1 or G2 is an antigen recognizing moiety, characterized in that AR is connected in the polymer chain via the 2,2′ or 3,3′ or 5,5′ or 6,6′ or 7,7′ or 8,8′ position according to general formula (II)

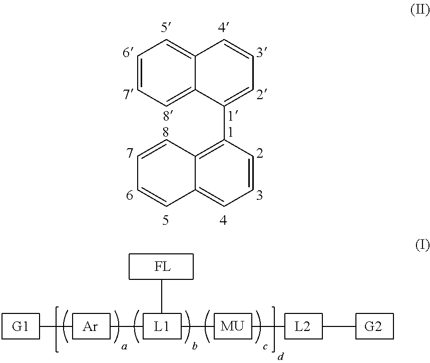

### BACKGROUND

The present invention is directed in general to fluorescent polymer conjugates and their use in methods of analyte detection.

Fluorescent probes are an important tool for the detection of biological analytes in life science applications. Fluorescent molecules attached to biological molecules such as antibodies are commonly used for immunostaining and sample analysis in an expanding array of applications ranging from flow cytometry, and fluorescence microscopy, to real-time PCR and genetic sequencing. With the expanding number of fluorescence-based applications comes a need for new bright, color-tunable, and water-soluble fluorescent molecules offering unique optical and physical properties which may aid in high multi-parameter specific analyte detection.

The usefulness of fluorescent molecules in multiplexed analyte detection applications depends highly upon the differentiating optical and physical properties of the molecule. Fluorescent Conjugated polymers (CPs) have been used recently for signal amplification and as bright fluorescent moieties in the detection of analytes as in U.S. Ser. No. 10/126,302B2 or U.S. Ser. No. 10/481,161B2. These inventions describe the structures of polyfluorene CP's and their utility in sensitive detection of analytes. Recent advances in instrumentation for multiplexed analyte detection, however, have demonstrated an additional need for fluorescent molecules that are not only sensitive, but also maintain controllable optical characteristics. Fluorescent molecules with an ability to individually tailor the excitation, emission, and light polarization properties are needed to enable a high level of -plexing and meet this growing need for unique fluorescence-based detection. To-date, however, no such molecules exist capable of combining all of these properties into a single molecule.

US20180009990A1 describes binaphthyl components substituted with PEGs in the 2,2′positions for use in water solvated polymeric dyes and polymeric tandem dyes. The polymeric dyes include a water solvated light harvesting multichromophore having a conjugated segment of aryl or heteroaryl co-monomers linked via polyparaethynylene (PPE) groups. The PPE-linked Binaphthyl polymer structures disclosed, however, are limited in color-tunability, water-solubility, and usefulness in analyte-sensing applications.

### OBJECT OF THE INVENTION

Objection of the invention is a conjugate or a dye having the general formula (I)

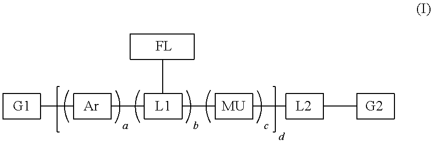

With AR, MU and L1 as repeating units of a polymer
MU is a polymer modifying unit or band gap modifying unit that is evenly or randomly distributed along the polymer main chain,
L1 is an aryl or a heteroaryl group evenly or randomly distributed along the polymer,
L2 is an aryl or a heteroaryl group located on the ends of the polymer,
FL is a fluorescent moiety,
G1 and G2 stand for hydrogen, halogen or an antigen recognizing moiety, with the provision that at least one of G1 or G2 is an antigen recognizing moiety,
a is 10 to 100 mol %,
b is 0.1 to 50 mol %
c is 0 to 90 mol %
d is 1 to 10,000; with the provisio that a+b+c=100 mol %
characterized in that AR is connected in the polymer chain via the 2,2′ or 3,3′ or 5,5′ or 6,6′ or 7,7′ or 8,8′ positions according to general formula (II)

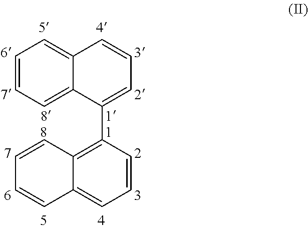

Wherein position 2,2′; 3,3′; 4,4′; 5,5′; 6,6′; 7,7′ and 8,8′ are same or different substituted with residues selected from the group consisting of H, SO₂CF₃, SO₂Rₐ, CF₃, CCl₃, CN, SO₃H, NO₂, NRₐR_{b}R_{c}⁺, CHO, CORa, CO₂Ra, COCl, CONRaRb, F, Cl, Br, I, Rₐ, ORₐ, SRₐ, OCORₐ, NRₐR_{b}, NHCORₐ, CCRₐ, aryl-, heteroaryl-, C₆H₄ORₐ or C₆H₄NRaRb, with Ra-c independently hydrogen, alkyl-, alkenyl-, alkinyl-, heteroalkyl-, aryl-, heteroaryl-, cycloalkyl-, alkylcycloalkyl-, heteroalkylcycloalkyl-, heterocycloalkyl-, aralkyl- or a heteroaralkyl residue or (CH₂)x(OCH₂CH₂)yO(CH₂)zCH₃,
x is an integer from 0-20 and
y is an integer from 0 to 50;
z is an integer from 0-20,
or two residues both as part of a cycloalkyl- or heterocycloalkyl ring system.

Conjugates of the invention are preferable water-soluble.

The term “AR” is connected in the polymer chain via the 2,2′ or 3,3′ or 5,5′ or 6,6′ or 7,7′ or 8,8′ positions” refers to C—C bonds between the respective C atoms of the positions of AR with a C-atom of another AR unit, a G1 unit or an L1unit.

Further object of the invention is a method comprising the steps
a) contacting the sample of biological specimens with at least one conjugate (I), thereby labeling the target moiety recognized by an antigen recognizing moiety with the conjugate (I);
b) exciting the labelled target moieties with light having a wavelength within the absorbance spectrum of the multichromophore CP-FL or the fluorescent moiety FL;
c) detecting the labelled target moieties by detecting the fluorescence radiation emitted by the multichromophore or the fluorescent moiety FL.

Yet another object of the invention is the use of the method in fluorescence microscopy, flow cytometry, fluorescence spectroscopy, cell separation, pathology or histology.

### BRIEF DESCRIPTION OF THE DRAWINGS

FIG. 1 shows normalized absorption (A) and emission spectra (B) recorded in Phosphate Buffered Saline (PBS) buffer for example copolymeric dyes spanning the UV and visible light region.

FIG. 2 shows examples of possible polymerization positions depicted with asterisks (*), and possible substitution patterns depicted with “R”.

FIG. 3a and FIG. 3b show a schematic example of binaphthyl based polymers with either R- or S-chirality of the monomer leading to chiral polymers with emission of circular polarized light, which can be modified by incorporation of bandgap modifying units (MU).

FIG. 4 shows a schematic representation of this invention strategy for bathochromic shifting of the dye fluorescence emission from the UV region to a target region

FIG. 5 (A) A schematic representation of two chiral dyes based on either R- or S-chiral monomer with different circular polarized luminescence upon excitation with the same excitation wavelength. (B) An example schematic representation for the detection of cells in a microfluidic platform, in which the cells are labeled with a dye that emits at the same wavelength, but in which the fluorescence emission is distinguishable based upon their circular polarization as an additional parameter in multiplexed analysis.

### DETAILED DESCRIPTION

In the following, the conjugate (I) is referred to CP-FL with polymer backbone CP as defined as followed,

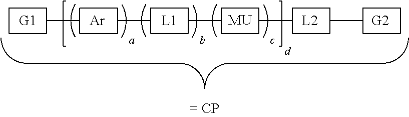

to which one or more fluorescent moieties FL are attached to L1 in accordance with formula (I)

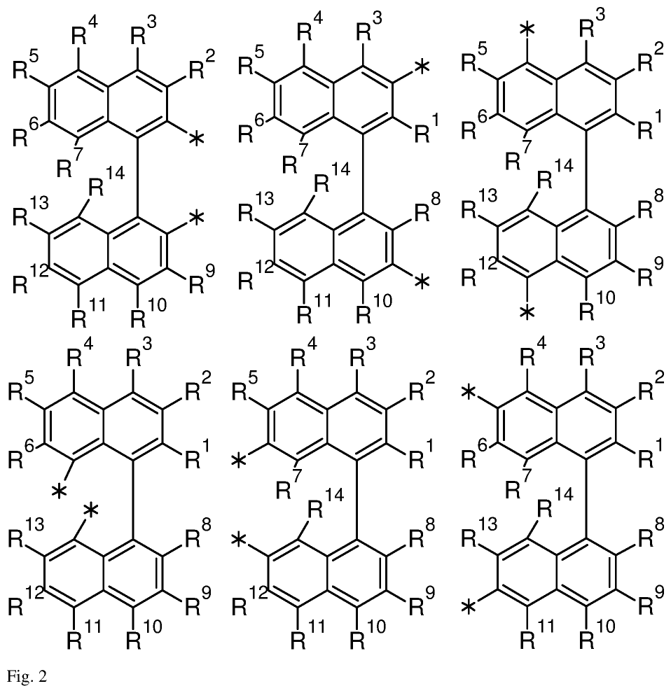

AR may be connected in the polymer chain via one of the 2,2′ or 3,3′ or 5,5′ or 6,6′ or 7,7′ or 8,8′ positions according to general formula (II) as shown in FIG. 2.

The respective other positions i.e. one or more of the remaining positions 2,2′; 3,3′; 4,4′; 5,5′; 6,6′; 7,7′ and 8,8′ are substituted with the same or different residues selected from the list as disclosed. In a preferred variant only one of the pairs of positions 2,2′; 3,3′; 4,4′; 5,5′; 6,6′; 7,7′ or 8,8′ is provided with residues selected from the list as disclosed. In a more preferred variant, only one of the pairs of positions 2,2′; 3,3′; 4,4′; 5,5′; 6,6′; 7,7′ or 8,8′ is provided with the same residue.

More preferable, the conjugate according to the invention is substituted at least at one pair of positions 2,2′; 3,3′; 4,4′; 5,5′; 6,6′; 7,7′ or 8,8′ with residues according to general formula (III)

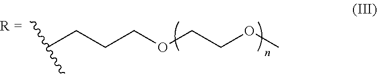

With n=5 to 15.

Most preferred is a conjugate substituted at one pair of positions 2,2′; 3,3′; 4,4′; 5,5′; 6,6′; 7,7′ or 8,8′ with residues according to general formula (III) having n=11.

MU is a polymer modifying unit or band gap modifying unit that is evenly or randomly distributed along the polymer main chain and is optionally substituted with one or more optionally substituted substituents selected from halogen, hydroxyl, C1-C12 alkyl, C2-C12 alkene, C2-C12 alkyne, C3-C12 cycloalkyl, C1-C12 haloalkyl, C1-C12 alkoxy, C2-C18 (hetero)aryloxy, C2-C18 (hetero) arylamino, a C2-C18 (hetero)aryl group and (CH2)x,(OCH2CH2)yO(CH2)zCH3 where
x is an integer from 0-20 and
y is an integer from 0 to 50;
z is an integer from 0-20.

L1 is an aryl or a heteroaryl group evenly or randomly distributed along the polymer main chain and is substituted with one or more pendant chains terminated with: i) a functional group selected from amine, carbamate, carboxylic acid, carboxylate, maleimide, activated ester, N-hydroxysuccinimidyl, hydrazine, hydrazide, hydrazine, azide, alkyne, aldehyde, thiol, and protected groups thereof for conjugation to a molecule or biomolecule; or ii) an attached conjugated organic fluorescent dye as energy acceptor of an energy transfer system, or iii) a biomolecule.

L2 is an aryl or a heteroaryl group located at the ends of the polymer main chain and is substituted with one or more pendant chains terminated with: i) a functional group selected from amine, carbamate, carboxylic acid, carboxylate, maleimide, activated ester, N-hydroxysuccinimidyl, hydrazine, hydrazide, hydrazine, azide, alkyne, aldehyde, thiol, and protected groups thereof for conjugation to a molecule or biomolecule; or ii) an attached organic fluorescent dye as energy acceptor of an energy transfer system, or iii) a biomolecule.

Preferable, L2 is selected from one or more of the following structures:

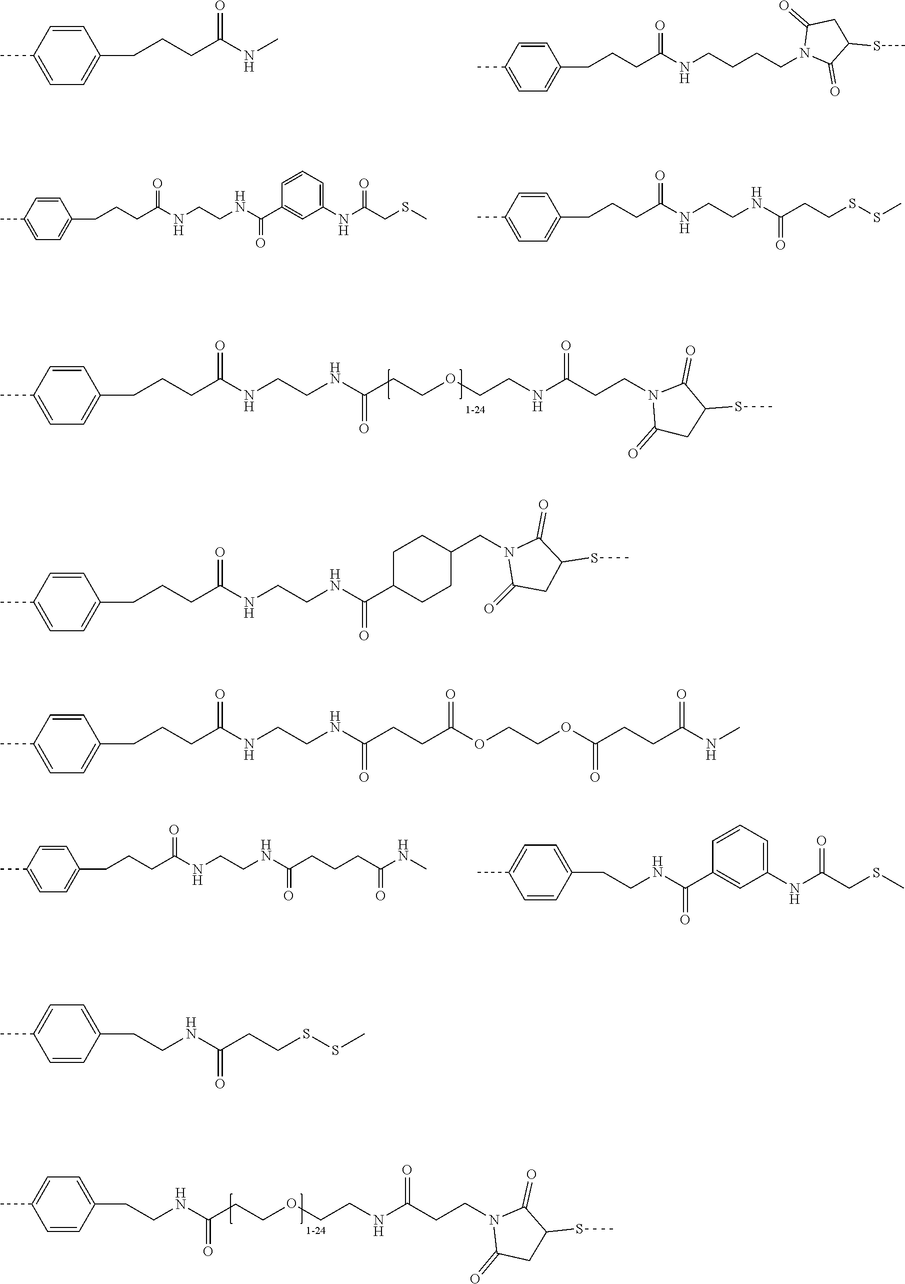

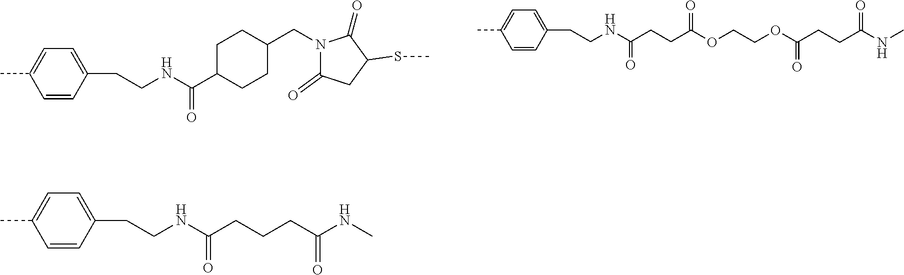

Preferred examples of the conjugate of the invention are shown in formula (IV) and (V)

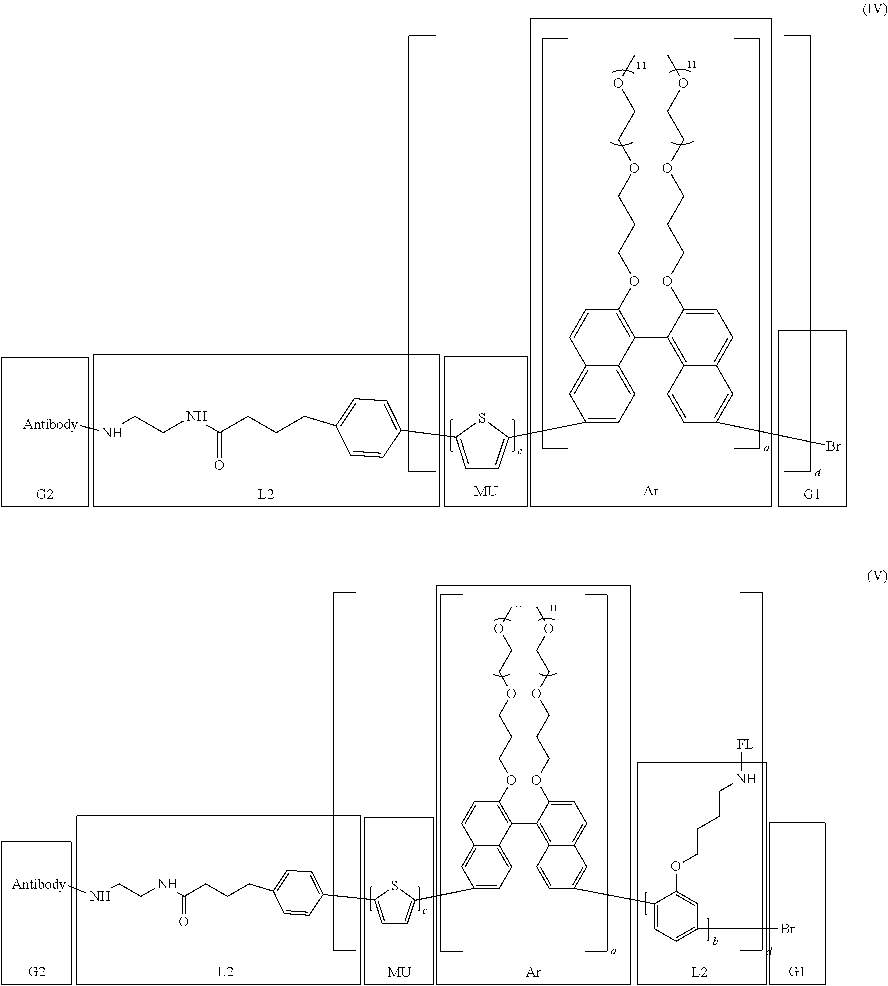

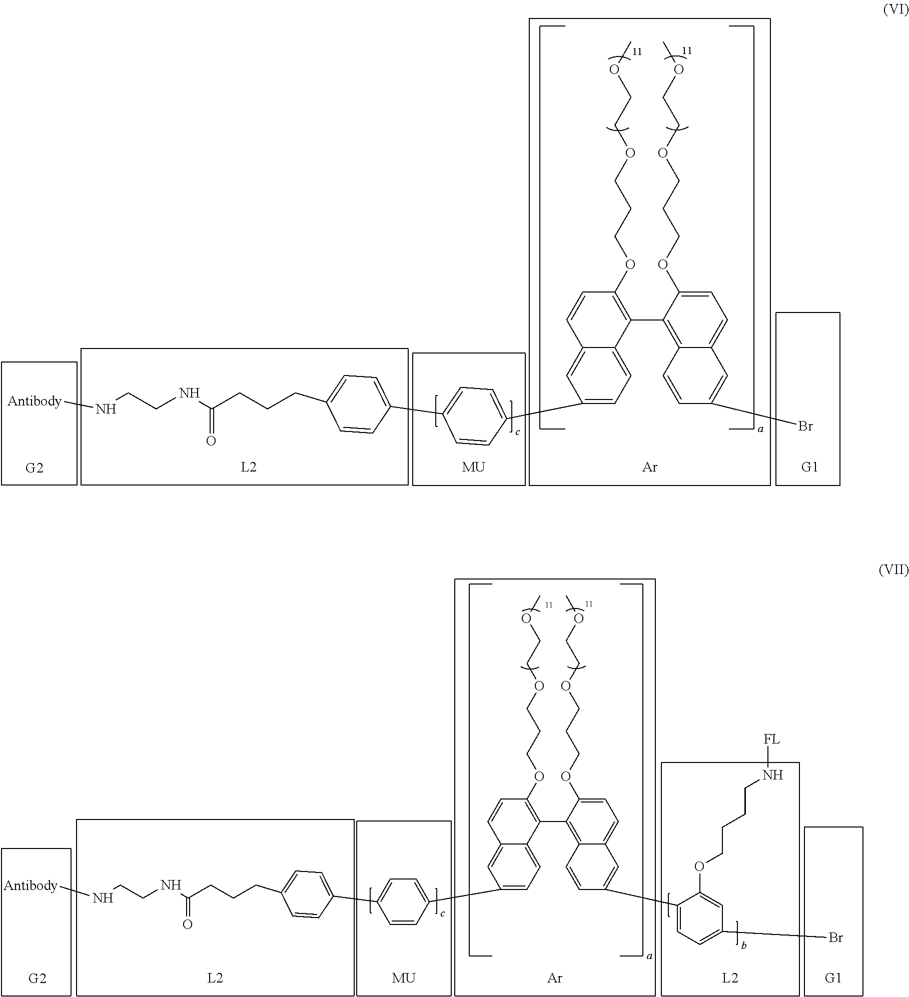

In recent years, various life science research areas including bioinformatics, immunological research, and drug discovery, have accelerated the trend toward increasing the number of analytes needed to be measured within a single experiment. In response to this demand for higher multiplexing ability in the research market, a host of innovative instruments has arrived capable of distinguishing previously indistinguishable fluorescent molecules based on differences in excitation as well as emission properties. Spectral flow cytometry and spectral analyzers in microscopy are two such examples of these instruments, although the need is not limited to only these instrument categories.

The introduction of spectral instruments for highly-multiplexed analyte detection and analysis has created an opportunity for novel fluorescent molecules to be developed. The ideal fluorescent molecule technology should be capable of addressing excitation energies from the “deep” Ultraviolet (280 nm) through the ultraviolet (˜350 nm), the violet (405 nm), and into the visible region of the spectrum in a controllable and color-tunable fashion, while maintaining usefulness in the sensitive detection of dim or rare events in heterogenous cell or tissue samples.

Existing fluorescent molecular technologies which have been used to address this need to-date include:
Small molecule organic dyes—these are color-tunable and have a wide-variety of options, however they suffer from low absorption strength (extinction coefficients), especially when excited in the violet and ultraviolet spectrum, and are limited in brightness.
Phycobiliproteins—Light-harvesting multi-chromophore proteins extracted from natural products. These fluorescent molecules suffer from rapid photobleaching, they are not color-tunable on their own, have low extinction coefficients in the Violet and Ultraviolet spectrum, and can show inconsistent performance due to their source as a natural product.
Semiconductor Nanoparticles (Quantum Dots)—These inorganic materials are light harvesting multi-chromophores, color-tunable, and very bright in certain applications.
Quantum dots, however, suffer from general toxicity due to their heavy-metal composition, they are bulky and typically much larger than the targeting molecule they are attached to. Quantum dots also tend to exhibit high non-specific binding on cells and tissue samples.
Conjugated Polymers (CPs)— These are light-harvesting multichromophoric organic fluorophores consisting of a series of pi-conjugated optical units, with an ability to transfer energy to acceptor dyes for limited color-tunability. Currently known CPs, however, suffer from limited tunability of their excitation spectrum, have a tendency to undergo polymer stacking and aggregation in solution due to pi-pi interactions which leads to lower brightness and solubility.

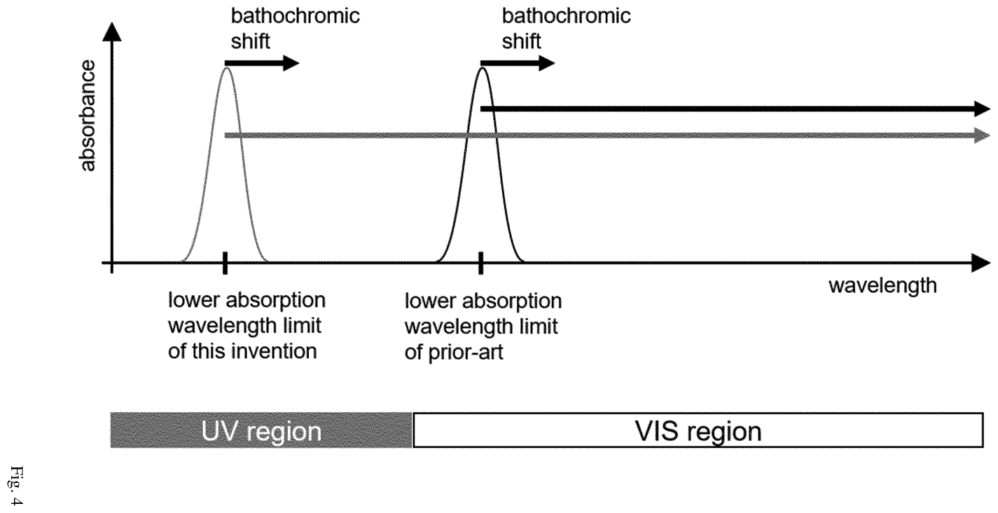

To overcome these limitations of the prior art, a material is required that shows the following characteristics:
Bright fluorescence: A high extinction coefficient and high fluorescence quantum yield provides materials with bright fluorescence signal, allowing detection of rare events and giving higher sensitivity in fluorescence analyte detection.
Water-solubility: High solubility in aqueous solutions is required for compatibility with biomolecules and biological materials like cells.
Color-tunability: A material whose absorption/emission bands and Stokes' shifts can be controlled, gives access to materials with unique photophysical properties. The ability to controllably shift absorption/emission bands from the UV region towards the violet region (bathochromic shift), as shown in FIG. 4, enables applications with UV/violet or longer wavelength lasers. In addition, individual control over each property: Absorption, Emission, and Stokes' shift as related to the bathochromic shift, provides distinctiveness to the material compared to materials naturally absorbing/emitting in the same spectral region.
Unique photophysical properties: Controllable excitation, emission and Stokes' shifts properties together with unique excitation/emission band profiles and/or chiroptical properties will enable multi-parameter detection.
Low self-aggregation: Because materials (in particular biomolecule conjugates) lose function and usefulness if they aggregate, materials with low self-aggregation are superior.

The prior art based on ethynylene-based polymers, as mentioned in US20180009990A1, does not meet the stated needs, and lacks compatibility with UV lasers. As illustrated in FIG. 4, for CPs of the prior art, the UV region of the spectrum is inaccessible via bathochromic shift. This limitation narrows their usable range down to only the violet, or longer, wavelength regions. The material in the mentioned prior-art cannot be used to address the UV region. To target the UV region using current CPs, a hypsochromic shift is required, but any spectral feature which is based on a bathochromic shift cannot therefore be achieved (e.g. a large Stokes' shift, broader widths of absorption/emission bands, etc). In this way, the lack of possibility to shift the absorption/emission bathochrome to the UV region prevents access to these unique photophysical properties.

Another limitation of the ethynylene-based polymers is the lack of options to introduce water-solubility. Attachment of a solubilizing group to the ethynylene unit is not possible, which leads to a material with poor water-solubility.

The exclusive use of racemic binaphthyl monomers from the prior art further excludes applications based on chiroptical properties, and therefore disregards another parameter for applications in multi-parameter detection.

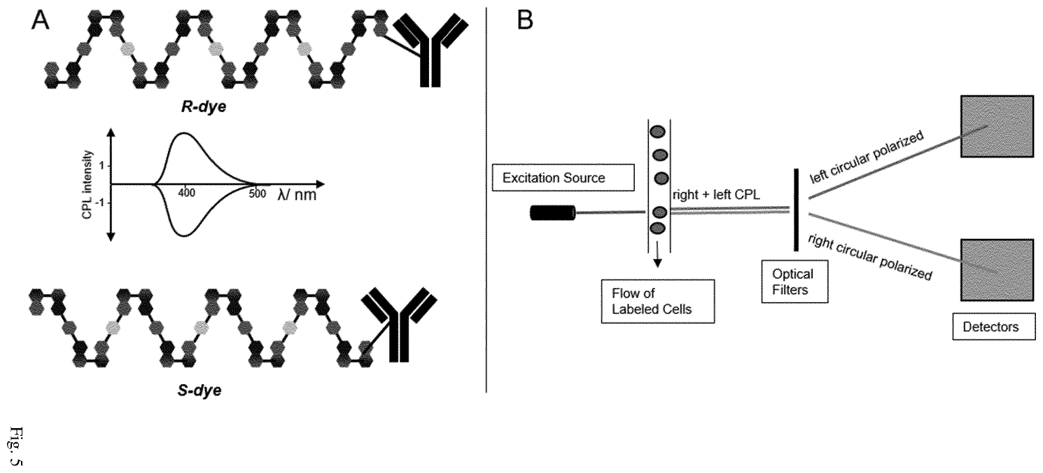

The material of this invention can serve all of the needs for color-tunability, water-solubility, and full control over photophysical properties. The CP of this invention is highly fluorescent and water-soluble, making it an ideal material for fluorescence based detection. The absorption in the UV region (e.g. at 280 nm), which can be shifted towards longer wavelengths (bathochromic shift) makes the new materials highly useful in combination with commercial UV (280 nm, 320 nm, 355 nm) or violet lasers (405 nm). Besides the ability to adjust the absorption band to the light source, a bathochromic shift allows the addition of unique photophysical properties, such as increasing the Stokes' shift by incorporation of comonomers (e.g. the incorporating of band-gap modifying units). Polymers based on enantiomerically pure monomers have unique chiroptical properties and these properties add another parameter for multi-parameter detection, as illustrated schematically in FIG. 5. Finally, since the materials of this invention also show low self-aggregation by controlling the angle between neighboring naphthyl groups, (i.e. the polymers are not planar and do not exhibit pi-pi stacking), the CP of this invention is ideal to serve all of the stated needs.

In the fluorescent polymer design and development of the invention, different concepts are applied to tune the polymer's spectral properties. The first concept is based on modification of the binaphthyl electron density. By introduction of a propyl ether, which is directly bound with the oxygen to the aromatic system, we have introduced an electron donating substituent which causes an increase of the binaphthyl electron density. That propyl ether carries a polyethylene glycol (PEG) chain to the binaphthyl group, which acts as a solubilizing group to provide water-solubility of the polymer. Selecting the 2,2′-positions for the propyl ether substitution has several advantages. One advantage is the possibility use the commercially available and cheap 1,1′-Bi-2-naphthol (BINOL) as starting material for the polymer synthesis.

The second concept is based on controlling the spatial polymer structure. An inherent property of the 1,1′-binaphthyl structure is a steric repulsion of the protons or substituents in the 8,8′-positions, that leads to a dihedral angle between the naphthyl groups typically in the range of 70-110°. This breaks the overall aromatic system and leads to optically distinguishable enantiomers. In order to stabilize the 1,1′-binapthyl from racemization, the PEG-carrying propyl ether substituents were introduced in the 2,2′-positions. The use of 1,1′-Bi-2-naphthol (BINOL) allows an easy and regioselective functionalization of the binaphthyl system at the 6,6′ positions, because of the electronic activation of the 6,6′-positions for reactions with electrophiles, e.g. in a bromination reaction using elemental bromine as the simplest bromination reagent.

Polymerization of binaphthyl monomers AR only through the 6,6′ position yields polymers as compound 1 in table 1. This compound absorbs at 315 nm in aqueous phosphate-buffered saline (PBS) buffer.

To shift this absorption to higher wavelengths, e.g. to absorb at 350 nm of a ultraviolet excitation source, or 405 nm of the commonly used violet laser, it is necessary to introduce bandgap modifying units into the polymer. Here, electron rich monomers can be combined with electron poor monomers, to create low band gap and therefore redshifted emitting polymers.

If this band gap is too low the, the polymer will absorb in the far red region. So for tuning the absorption of a conjugated polymer to fit the violet laser, it is possible to break the linearity of the molecule and therefore the electronic-conjugation along the polymer, by introducing kinks in the polymer chain. For this purpose, conjugates comprising binaphthyl-based polymers are preferred.

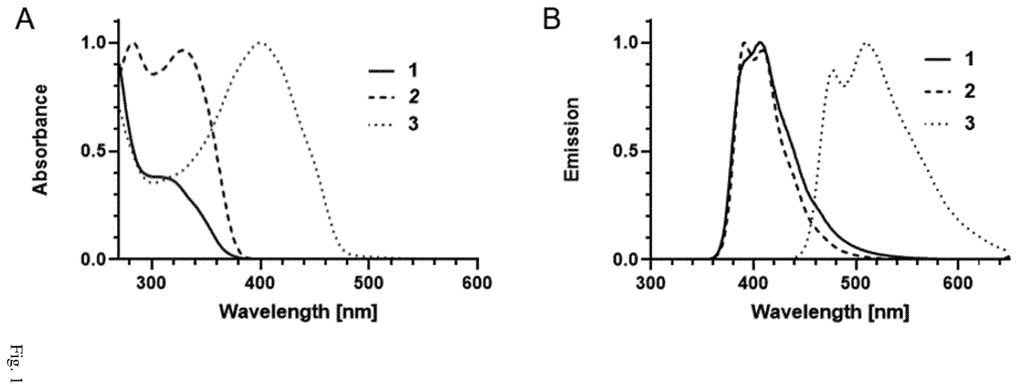

By combining an electron rich monomer (bithiophene) with the mainly electron neutral binaphthyl which is slightly electron rich because of the donating function of the phenolic ether group, the polymer absorbs at 400 nm and is therefore suited for a violet laser (table 1, compound 3). While combining the binaphthyl monomer with phenylene as a bandgap modifying unit will yield polymers suitable for UV lasers (table 1, compound 2). The absorption and emission spectra of these compounds are also shown in FIG. 1.

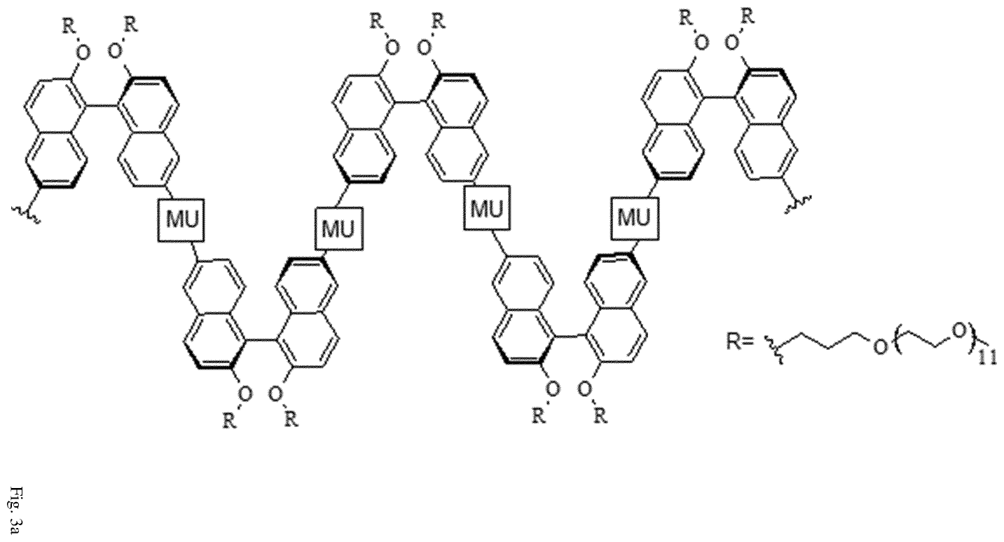

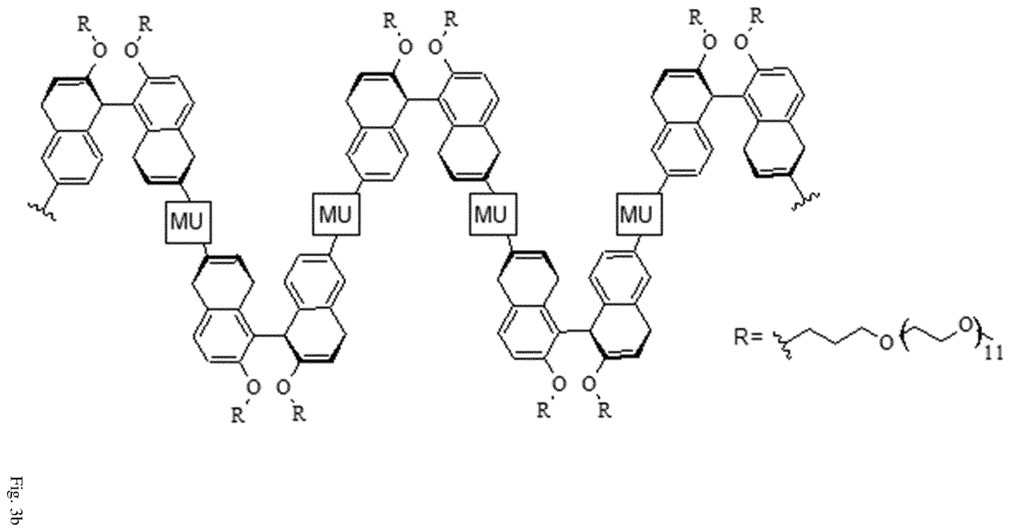

Further advantage of using binaphthyl monomers for π-conjugated fluorescent polymers is the inherent availability of R- and S-stereo isomers as building blocks for chiral-conjugated polymers as shown in FIG. 3.

In a preferred embodiment, the conjugate according to the invention has a main chirality with AR units provided mainly as R- or S-stereo isomer. Preferably, by using AR units polymerized through the 6,6′ positions, conjugates of the invention with a main chain chirality can be obtained. “Mainly” means more than 50% of the AR units are provided as R- or S-stereo isomer.

In a preferred variant, more than 75% or more than 90% of the AR units are provided as R- or S-stereo isomer. Obviously, in a most preferred version all (100%) of the AR units are provided as R- or S-stereo isomer.

The term “main chirality” refers to the overall interaction of the conjugates with polarized light. optical isomer does the opposite. If on an aqueous solution of the conjugate a rotation of light is measured using a polarimeter, the conjugate shows a “main chirality”.

The main chain chirality leads to unique chiroptical properties that can be used to distinguish fluorochromes absorbing or emitting at the same wavelength, but absorbing or emitting light with different circular polarization. By using this effect together with suitable detection set up using polarized filter sets it is possible to improve multiplexed detection in immunofluorescent applications.

### Method of the Invention

In the method of the invention, the labelled target moieties with light having a wavelength within the absorbance spectrum of the fluorescent moiety FL and the labelled target moieties are detected by detecting the fluorescence radiation emitted by the fluorescent moiety FL or CP. Possible light sources for excitation are commonly used lasers or LEDs in the ultraviolet, violet, blue, green/yellow, red, far red and near infrared region, that can for example be, but are not limited to, the following wavelengths of 280, 320, 355, 365, 375, 405, 488, 640, or 750 nm. The detection of the emission of the sample are the corresponding channels that suit the emission of either the polymer backbone CP or the emitting moiety in the CP-FL construct. Corresponding detection channels can be, for example but are not limited to 379/28, 450/50, 525/50, 579/34, 615/20, 667/30, 725/40, 785/62. Detection channels for CP-FL constructs can be chosen according to the emitting moiety, e.g. Fluorescein or Fluorescein-Derivatives, Rhodamine, Tetramethylrhodamine, Silicon-Rhodamine (SiR), Coumarines, Resorufines, Pyrenes, Anthracenes, Phenylenes, Phthalocyanines, Cyanines, Xanthenes, Amidopyrylium-Farbstoffe, Oxazine, Quadrain-Farbstoffe, Carbopyronine, 7-Nitrobenz-2-Oxa-1,3-Diazol (NBD) Fluorophore, BODIPY™ Fluorophores (Molecular Probes, Inc.), ALEXA T™ Fluorophore (Molecular Probes, Inc.), DY™ Fluorophores (Dyomics GmbH), Benzopyrylium Fluorophores, Benzopyrylium-Polymethin Fluorophores, Lanthanide-Chelate, Metalloporhyrines, Rhodol dyes, Carborhodol dyes, Naphthalimides and Porphyrins.

In a preferred embodiment of the method, after step c) is performed, the fluorescent moiety FL of the labelled target moieties is degraded by irradiating the conjugate with light having a wavelength within the absorbance spectrum of fluorescent moiety FL for a time sufficient to deliver enough energy to reduce the fluorescence radiation emitted by the fluorescent moiety FL at least by 75% of the initial fluorescence radiation.

In the method of the invention, the sample is irradiated with light having a wavelength within the absorbance spectrum of the fluorescent moiety FL in order to reduce the fluorescence radiation emitted by the fluorescent moiety so much that any residual fluorescence radiation from a first staining cycle does not interfere with subsequent staining and detection cycles. In general, reduction by at least 75% of the initial fluorescence radiation is deemed sufficient, but in order to achieve a higher quality of detection i.e. to reduce background radiation not originating from the staining step of interest, it is preferred to reduce fluorescence radiation by at least by 85%, more preferred at least by 95% and most preferred by at least 99%. While a reduction of 100% would be best, there is a trade-off with quenching quality and overall process duration.

In an alternative definition, degrading the fluorescent moiety FL attached to a conjugated polymer (CP) of the labelled target moieties is performed by irradiating the conjugate with light having a wavelength within the absorbance spectrum of fluorescent moiety FL or of CP or both (e.g. using white light) for a time sufficient to deliver enough energy to reduce the half-life of the fluorescence radiation emitted by the fluorescent moiety. The degradation rate given by the value of k from the mono-exponential decay fit analysis of the fluorescent moiety FL be at least 1.02 and up to 10.000.000 fold higher compared to the k obtained for the same fluorescent moiety non-conjugated to the conjugated polymer (CP).

Fluorescent moiety FL and antigen recognizing moiety can be bound covalently or quasi-covalently to CP. The terms “covalently or quasi-covalently” refers to the bonds between FL and CP and the antigen-recognizing moiety having a dissociation constant of greater or equal than 10-9 M.

The process of the invention may be performed in one or more sequences of the steps a) to c). After each sequence, the fluorescent moiety is degraded by irradiation with light. The terms “degrading”, “quenching” or “bleaching” are used interchangeably herein, and should be understood to mean the diminution of fluorescence intensity from the labeled biological sample, as result of an alteration of the fluorophore by radiation. For example, “quenching” or “bleaching” of the fluorescent moiety FL may be achieved by oxidation initiated by the radiation and/or by cleaving the fluorescent moiety FL from CP and removing the unbound fluorescent moiety from the labelled target by washing.

The bleaching system used in the present invention may be provided with more than one light sources emitting radiation of different wavelengths. For example the bleaching system may be provided with 1-5 light sources which have a combined emission spectrum in the range of 350-850 nm, preferable 400-650 nm. The emission of the light sources may optically combined to irradiate the sample simultaneously or subsequently. For example, the bleaching system may be provided with four light sources emitting in the ranges 380-410 (violet), 450-500 nm (blue), 520-560 nm (green) and 630-650 nm (red). In another embodiment only one light source is provided, emitting light in the range 200-1000 nm (white light), preferable 350-850 nm, and most preferable 400-650 nm. The advantage of separate light sources is that the sample is exposed to radiation only necessary to bleach (eliminate) the fluorescence dye thereby avoiding unnecessary exposure of the sample to radiation with other wavelengths. The radiation of the separate light sources may be combined by appropriate devices like mirrors or optical waveguide like optical fiber.

After and/or before each sequence, a washing step may be performed to remove unwanted material like unbound conjugates moieties and/or unbound fluorescent moieties FL from the sample.

The bleaching process as described may be further enhanced by adding oxidative agents. Oxidative agents may be for example 02, H₂O₂, peroxides or DMSO. The oxidative agents added may generate the active oxidative species, which, calculated as 0, should be present in concentrations of 0.1 to 5 ppm, preferable 2 to 5 ppm.

### Target Moiety

The target moiety to be detected with the method of the invention can be on any biological specimen, like tissues slices, cell aggregates, suspension cells, or adherent cells. The cells may be living or dead. Preferable, target moieties are antigens expressed intracellular or extracellular on biological specimen like whole animals, organs, tissues slices, cell aggregates, or single cells of invertebrates, (e.g., Caenorhabditis elegans, Drosophila melanogaster), vertebrates (e.g., Danio rerio, Xenopus laevis) and mammalians (e.g., Mus musculus, Homo Sapiens).

### Fluorescent Moiety FL

Suitable fluorescent moieties FL are those known from the art of immunofluorescence technologies, e.g., flowcytometry or fluorescence microscopy. In the method of the invention, the target moiety labelled with the conjugate is detected by exciting the CP backbone or the fluorescent moiety FL or both and detecting the resulting emission (photoluminescence) of FL or CP.

Useful fluorescent moieties FL might be protein based, such as phycobiliprotein, small organic molecule dyes, such as xanthenes, like fluorescein, or rhodamines, cyanines, oxazines, coumarins, acridines, oxadiazoles, pyrenes, pyrromethenes, pyridyloxazole or metallo-organic complexes, such as Ru, Eu, Pt complexes. Besides single molecule entities, clusters of fluorescent proteins or small organic molecule dyes, as well as nanoparticles, such as quantum dots, upconverting nanoparticles, gold nanoparticles, dyed polymer nanoparticles can also be used as fluorescent moieties.

In another embodiment of the invention the target labelled with the conjugate is not detected by radiation emission, but by absorption of UV, visible light, or NIR radiation. Suitable light-absorbing detection moieties are light absorbing dyes without fluorescence emission, such as small organic molecule quencher dyes like N-aryl rhodamines, azo dyes, and stilbenes. In another embodiment, the light-absorbing fluorescent moieties FL can be irradiated by pulsed laser light, generating an photoacoustic signal.

In a variant of the invention, the fluorophore FL is substituted with one more water solubility imparting substituents selected from the group consisting of sulfonates, phosphonates, phosphates, polyethers, sulfonamides and carbonates. It is particularly advantageous to use fluorescent moieties with sulfonate substituents, such as dyes of the Alexa Fluor family provided by Thermo Fisher Scientific Inc. The degree of sulfonate substitution per fluorophore may be 2 or more, i.e., for rhodamine dyes or cyanine dyes.

Suitable commercial available fluorescent moieties may be purchased from the product line “Vio” from Miltenyi Biotec BV & Co. KG, or FITC, or Promofluor, or Alexa Dyes and/or Bodipy dyes from Thermofisher, or Cyanines from Lumiprobe or DY™ Fluorophore from Dyomics GmbH or Star Dyes from Abberior GmbH.

### Antigen Recognizing Moiety

The term “antigen recognizing moiety” refers to any kind of antibody, fragmented antibody or fragmented antibody derivatives, directed against the target moieties expressed on the biological specimens, like antigens expressed intracellular or extracellular on cells. The term relates to fully intact antibodies, fragmented antibody or fragmented antibody derivatives, e. g., Fab, Fab′, F(ab′)2, sdAb, scFv, di-scFv, nanobodies. Such fragmented antibody derivatives may be synthesized by recombinant procedures including covalent and non-covalent conjugates containing these kind of molecules. Further examples of antigen recognizing moieties are peptide/MHC-complexes targeting TCR molecules, cell adhesion receptor molecules, receptors for costimulatory molecules, artificial engineered binding molecules, e.g., peptides or aptamers which target, e.g., cellsurface molecules.

The conjugate used in the method of the invention may comprise up to 100, preferable 1-20 antigen recognizing moieties Y. The interaction of the antigen recognizing moiety with the target antigen can be of high or low affinity Binding interactions of a single low-affinity antigen recognizing moiety is too low to provide a stable bond with the antigen. Low-affinity antigen recognizing moieties can be multimerized by conjugation to the enzymatically degradable spacer to furnish high avidity. When the spacer is enzymatically cleaved, the low-affinity antigen recognizing moieties will be monomerized which results in a complete removal of the fluorescent marker.

Preferable, the term “Antigen recognizing moiety” refers to an antibody directed against antigen expressed by the biological specimens (target cells) intracellular, like IL2, FoxP3, CD154, or extracellular, like CD19, CD3, CD14, CD4, CD, CD25, CD34, CD56, and CD133. The antigen recognizing moieties G1, G2, especially antibodies, can be coupled to CP through side chain amino or sulfhydryl groups. In some cases the glycosidic side chain of the antibody can be oxidized by periodate resulting in aldehyde functional groups.

The antigen recognizing moiety can be covalently or non-covalently coupled. Methods for covalent or non-covalent conjugation are known by persons skilled in the art and the same as mentioned for conjugation of the fluorescent marker.

The method of the invention is especially useful for detection and/or isolation of specific cell types from complex mixtures and may comprise more than one sequential sequences of the steps a)-d). The method may use a variety of combinations of conjugates. For example, a conjugate may comprise antibodies specific for two different epitopes, like two different anti-CD34 antibodies. Different antigens may be addressed with different conjugates comprising different antibodies, for example, anti-CD4 and anti-CD8 for differentiation between two distinct T-cell-populations or anti-CD4 and anti-CD25 for determination of different cell subpopulations like regulatory T-cells.

### Cell Detection Methods

Targets labelled with the conjugate are detected by exciting either the fluorescent moiety FL or the backbone CP and analysing the resulting fluorescence signal. The wavelength of the excitation is usually selected according to the absorption maximum of the fluorescent moiety FL or CP and provided by LASER or LED sources as known in the art. If several different detection moieties FL are used for multiple colour/parameter detection, care should be taken to select fluorescent moieties having not overlapping absorption spectra, at least not overlapping absorption maxima. In case of fluorescent moieties the targets may be detected, e.g., under a fluorescence microscope, in a flow cytometer, a spectrofluorometer, or a fluorescence scanner. Light emitted by chemiluminescence can be detected by similar instrumentation omitting the excitation.

### Use of the Method

The method of the invention can be used for various applications in research, diagnostics and cell therapy, like in fluorescence microscopy, flow cytometer, fluorescence spectroscopy, cell separation, pathology or histology.

In a first variant of the invention, biological specimens like cells are detected for counting purposes i.e. to establish the amount of cells from a sample having a certain set of antigens recognized by the antigen recognizing moieties of the conjugate. In another variant, the biological specimens detected by the conjugate in step c) are separated from the sample by optical means, electrostatic forces, piezoelectric forces, mechanical separation or acoustic means. For this purpose, the biological specimens detected by the conjugate in step d) are separated from the sample according to their detection signal to one or more populations simultaneously or subsequent before performing step d) by optical means, electrostatic forces, piezoelectric forces, mechanical separation or acoustic means.

In another variant of the invention, the location of the target moieties like antigens on the biological specimens recognized by the antigen recognizing moieties of the conjugate is determined. Such techniques are known as “Multi Epitope Ligand Cartography”, “Chip-based Cytometry” or “Multiomyx” and are described, for example, in EP0810428, EP1181525, EP 1136822 or EP1224472. In this technology, cells are immobilized and contacted with antibodies coupled to fluorescent moiety. The antibodies are recognized by the respective antigens on the biological specimen (for example on a cell surfaced) and after removing the unbound marker and exciting the furescentieties, the location of the antigen is detected by the fluorescence emission of the fluorescent moieties. In certain variants, instead of antibodies coupled to fluorescent moieties, antibodies coupled to moieties detectable for MALDI-Imaging or CyTOF can be used. The person skilled in the art is aware how to modify the technique based on fluorescent moiety to work with these detection moieties.

The location of the target moieties is achieved by a digital imaging device with a sufficient resolution and sensitivity in for the wavelength of the fluorescence radiation, The digital imaging device may be used with or without optical enlargement for example with a fluorescence microscope. The resulting images are stored on an appropriate storing device like a hard drive, for example in RAW, TIF, JPEG, or HDF5 format.

In order to detect different antigens, different antibody-conjugates having the same or different fluorescent moiety or antigen recognizing moiety can be provided. Since the parallel detection of fluorescence emission with different wavelengths is limited, the antibody—fluorochrome conjugates are utilized sequentially individually or in small groups (2-10) after the other.

In yet another variant of the method according to the invention, the biological specimens especially suspension cells of the sample are immobilized by trapping in microcavities or by adherence.

In general, the method of the invention can be performed in several variants. For example, the conjugate not recognized by a target moiety can be removed by washing for example with buffer before the target moiety labelled with the conjugate is detected.

In a variant of the invention, at least two conjugates are provided simultaneously or in subsequent staining sequences, wherein each antigen recognizing moiety recognizes different antigens. In an alternative variant, at least two conjugates can be provided to the sample simultaneously or in subsequent staining sequences. In both cases, the labelled target moieties can be detected simultaneously or sequentially.

### Examples

Conjugated homo polymers using 6,6′-dibromo-2,2′-C3PEG11-binaphtyl monomers were polymerized using Yamamoto conditions and copolymers with 2,2′-bithiophene-5,5′-diboronic acid bis(pinacol) ester or 1,4-phenyldiboronic acid bis(pinacol) ester comonomers were polymerized using Suzuki conditions. The resulting polymers are of the general structure with the subunit MU being bithiophene shown iu formula (VIII) with n=0 (compound 1) and n=2 (compound 3) or for the phenylene as MU in structures VI (compound 2) and VII.

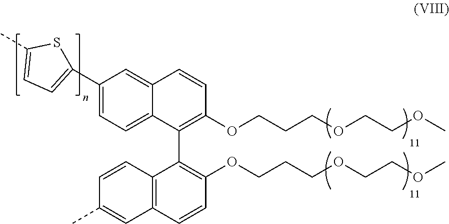

Absorption and emission spectra of the resulting polymers were recorded in PBS and methylene chloride and are shown in table 1 as well as their quantum yield and the molecular weight distribution results of gel permeations chromatography. GPC analysis was conducted using DMF with 0.01% lithium bromide as eluent. The data were calibrated against polystyrene standards.

Table 1 shows result for two different binaphtyl-based polymers. Compound 1 is a polybinaphthyl derivate of the conjugate without bandgap modifying units (MU) with n=0. This compound shows an quantum yield of 60% in aqeuous buffer. The absorption maximum is at 315 nm. This is suitable for deep UV excitation sources, but since in this region also many biomolecules absorb and this causes high cellular autofluorescence, spectral tuning to higher absorption and emission wavelengths is also interesting.

This can be easily done using binaphtyl-based fluorescent polymers, by introducing MU into the polymer chain, like phenylene or bithiophen, as in compound 2 (structure VI) and 3 (structure VIII, with n=2). Compound 2 has a good quantum yield of 33%, while compound 3 still shows a good quantum yield of 18%, which together with the intrinsic high extinction coefficient of fluorescent polymers, due to their extended π-electron system leads to an excellent brightness. The ladder polymer 3 now shows an optimal absorption at 400 nm, which makes the polymer suited for usage with the violet laser. See also FIG. 1. 0.5

TABLE 1
GPC results and photophysical properties of compounds 1, 2 and 3.

| | Mn/ | Mw/ | PBS | | |
|---|---|---|---|---|---|
| Polymer | kDa | kDa | Abs/nm | Em/nm | ϕfl |
| Poly(binaphthyl) 1 | 8 | 10 | 265, 315 | 390, 408 | 0.60 |
| Poly(binaphthyl-alt- | 19.1 | 33.8 | 330 | 406 | 0.33 |
| phenylene) 2 | | | | | |
| Poly(binaphthyl-alt- | 10 | 19 | (244), 400 | (477), 511 | 0.18 |
| bithiophen) 3 | | | | | |

### General Synthesis

### Capper Synthesis

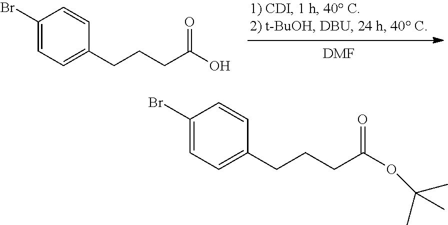

A solution of 4-(4-Bromophenyl)butanoic acid (1.00 g) and 1,1′-Carbonyldiimidazole (667 mg) in DMF (4.1 mL) was stirred for 24 h at 40° C. under argon. After addition of t-BuOH (610 mg) and 1,8-Diazabicyclo[5.4.0]undec-7-en the reaction mixture was stirred for another 24 h at 40° C. After addition of ether (50 mL) the solution was washed 10% hydrochloric acid (10 mL), water (10 mL) and aqueous 10% sodium carbonate (10 mL) and dried over sodium sulfate to give a brown oil. Silica column chromatography purification yielded the pure product (212 mg/17%).

### Synthesis of 1,1-Binaphthyl Monomer

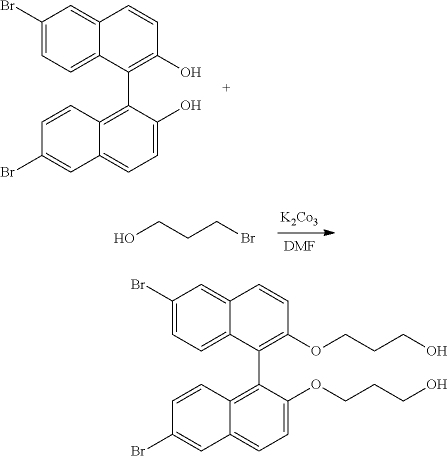

In a three-neck round bottom flask 6,6′-Dibrom-1,1′-bi-2-naphthol (24.43 g) and potassium carbonate (22.80 g) were stirred in DMF under argon and heated to 80° C. 3-bromopropanol (22.94 g) was added dropwise over 30 min. After stirring for 6 h at 80° C., water was added the mixture was extracted with ethyl acetate. The organic phase was dried over sodium sulfate and the solvent was evaporated. The crude product was purified by column chromatography to yield the product as white crystals (12.9 g, 42%).

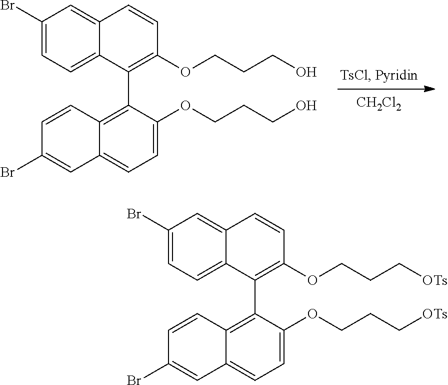

In a three-neck round bottom flask 6,6′-Dibrom-2,2′-bis-(3-hydroxypropoxy)-1,1′-binaphthalin (12.33 g) and tosyl chloride (12.58 g) were stirred in DCM (150 mL) under argon. The suspension was cooled down to 0° C. and pyridine was added dropwise. The mixture was stirred for 2 h at 0° C. and subsequently was warmed up to room temperature and stirred for 48 h. After adding water, the solvent was evaporated and the crude product was recrystallized two times from methanol to yield the product as white crystals (7.3 g, 38%).

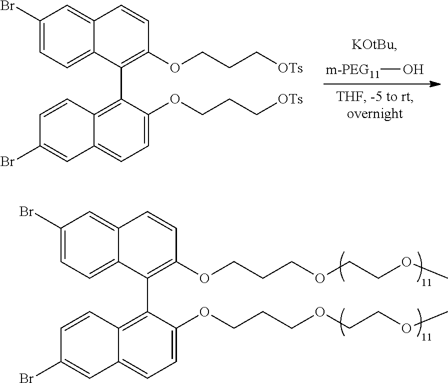

In a three-neck round bottom flask KOtBu (1.234 g) and m-PEG-11-OH (6.193) were stirred in THF under argon. The suspension was cooled to −5° C. and 6,6′-Dibrom-2,2′-bis-(3-tosyl-propoxy)-1,1′-binaphthalin was added in one portion. The mixture was stirred for 2 h at 0° C. and was subsequently warmed up to room temperature and stirred for 24 h. After diluting with THF the solution was filtered and the solvent was evaporated to yield an oil. The crude product was purified by column chromatography (normal+reverse phase) to yield the product (3.08 g, 49%) as a yellow oil.

### Polymerization via Yamamoto conditions

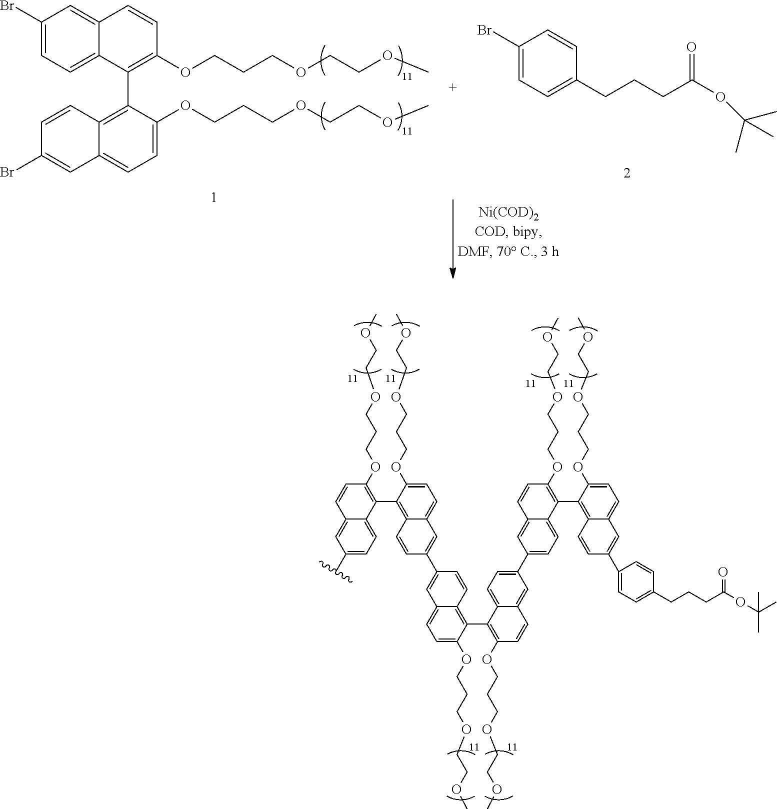

Bis(1,5-cyclooctadienyl)nickel(0) (97.2 mg), 2,2′-bipyridyl (55.2 mg) and 1,5-cyclooctadiene (38.2 mg) were added to a 50 mL round bottom flask and were stirred in DMF (8 mL) under argon for 30 min at 70° C. Compound 1 (250 mg) and 2 (1.23 mg) were dissolved in DMF (4 ml) and added to the reaction mixture. The solution was stirred for 3 h at 70° C. The solvent was evaporated and the residue was suspended in ethanol (20%, aq.). After centrifuging for 60 min at 13000×g the supernatant was freeze dried to yield the product (101 mg, 45%).

### Polymerization via Suzuki conditions

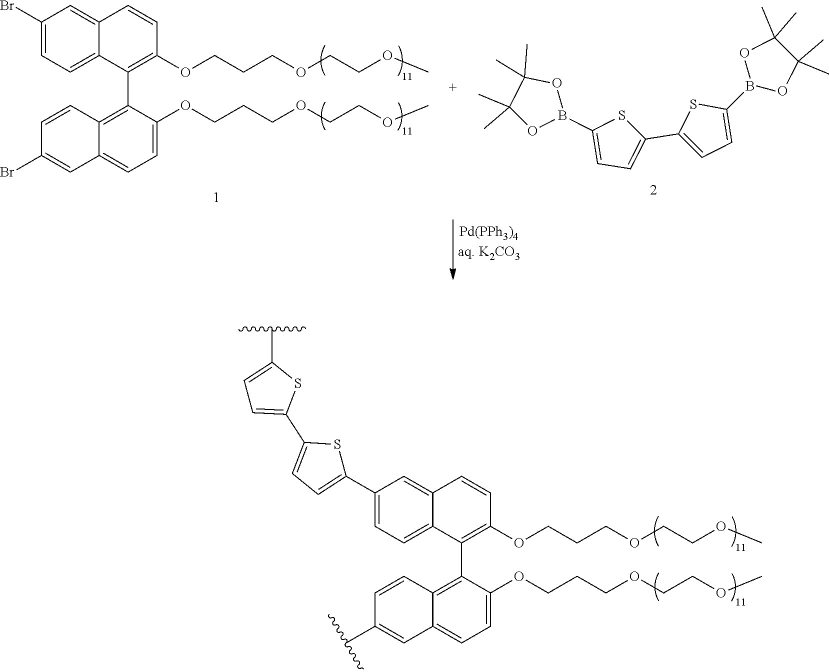

To a mixture of compound 1 (250 mg) and 2 (67 mg) in DMF (4 mL) in a Schlenk-flask, Pd(PPh₃)₄ was added under argon. Aqueous K₂CO₃ (2 M, 750 μL) was added and the solution was degassed by using 3 freeze-pump-thaw cycles. Afterwards the solution was heated to 80° C. for 3 h. The solvent was evaporated and the residue was suspended in ethanol (20%, aq.). After centrifuging for 60 min at 13000 g the supernatant was freeze dried to yield the amber product (120 mg, 48%).

### Polymerization via Suzuki Conditions

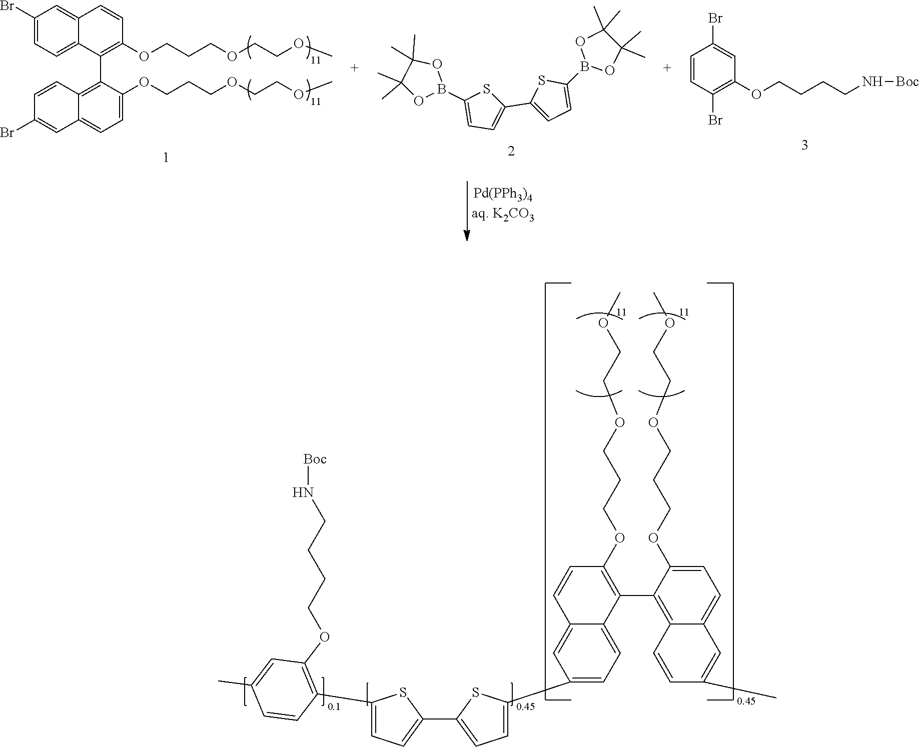

To a mixture of compound 1 (250 mg) and 2 (67 mg) and 3 (6.7 mg) in DMF (4 mL) in a Schlenk-flask, Pd(PPh3)4 was added under argon. Aqueous K2CO3 (2 M, 750 μL) was added and the solution was degassed by using 3 freeze-pump-thaw cycles. Afterwards the solution was heated to 80° C. for 3 h. The solvent was evaporated and the residue was suspended in ethanol (20%, aq.). After centrifuging for 60 min at 13000 g the supernatant was freeze dried to yield the product.

### Deprotection of Polymer Backbone

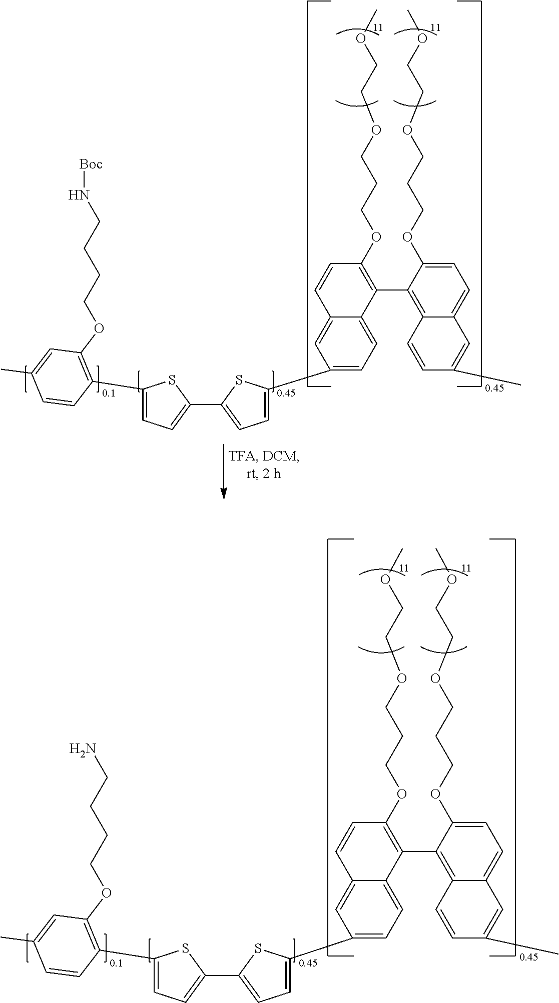

The polymer (329 mg) was dissolved in DCM (50 mL) and trifluoroacetic acid (5 mL) was added. The solution was stirred for 2 h at room temperature. The solvent was evaporated and the residue was dissolved in aqueous ethanol (20%, 100 mL), purified by size exclusion centrifugal filtration (10 kDa cutoff) and freeze dried to yield the final product (290 mg).

### Tandem Dye Synthesis

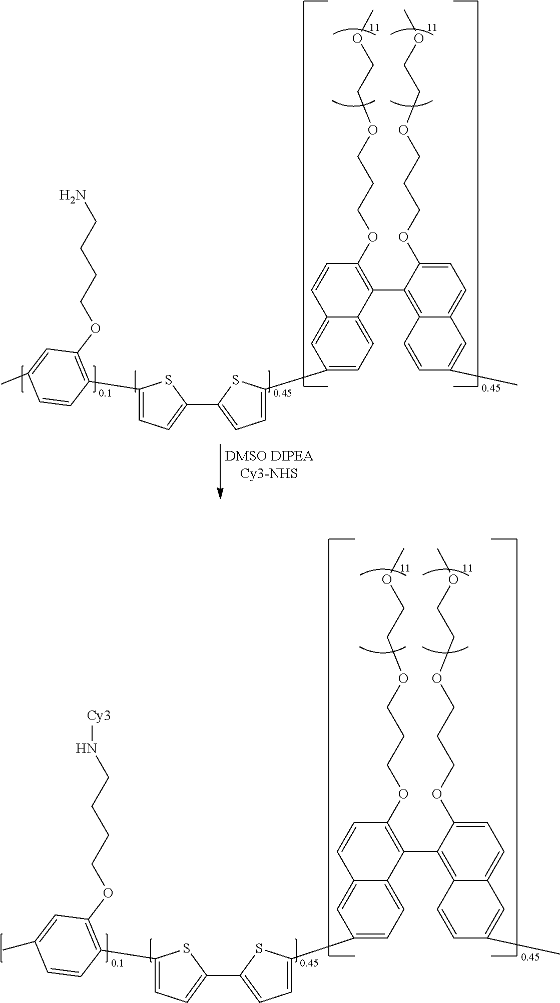

The polymer (2 mg) was dissolved in DMSO (133 μL) in a screw cap vial under argon. After addition of DIPEA (8.2 mg) and Cy3-NHS (1 mg) the solution was stirred for 16 h. The solution was diluted with aqueous ethanol (20%, 40 mL) and purified by size exclusion centrifugal filtration (10 kDa cutoff). Freeze drying yields the product as a pink solid.

### Conjugation to a Primary Antibody Via Primary Amines

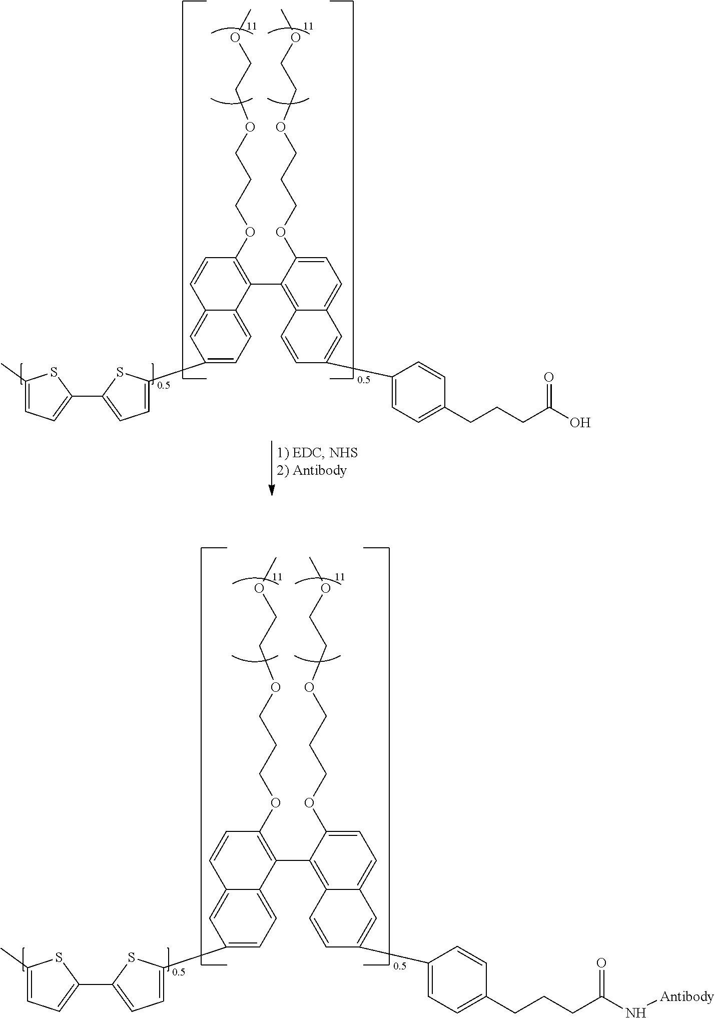

The polymer (2 mg) was dissolved in 200 mM MES buffer (100 μl) in an 1.5 mL Eppendorf cap (50 mg/mL). N-Hydroxysuccinimid (11.9 mg) and N-(3-dimethylaminopropyl)-N′-ethylcarbodiimide hydrochloride (11.2 mg) were dissolved in (497 μL) MES buffer in an 1.5 mL Eppendorf cap. 40 μL of the solution was added to the polymer solution and incubated 15 5 min at room temperature while shaking at 800 rpm using a ThermoMix. The activated polymer was purified via an size exclusion filtration with molecular weight cut-off of 10 kDa. CD4 antibody (0.5 mg) was dissolved in PBS buffer (921 μL) in an 2 mL Eppendorf cap, after adding the purified activated polymer 0.5 M carbonate buffer (125 μL) to the antibody, the mixture was incubated 2.5 h at room temperature and shaking at 800 rpm. The mix was concentrated size exclusion filtration with molecular weight cut-off of 10 kDa. The conjugate was purified via size exclusion chromatography using PBS buffer as eluent. The antibody-polymer conjugates were eluting prior to the free antibody, allowing good separation. Different fractions were pooled to yield the final antibody-polymer conjugate.

1. A conjugate having the general formula (I) 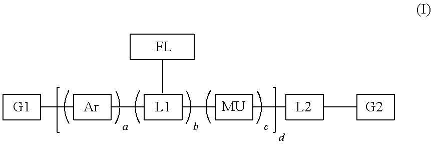
With AR, MU and L1 as repeating units of a polymer
MU is a polymer modifying unit or band gap modifying unit that is evenly or randomly distributed along the polymer main chain,
L1 is an aryl or a heteroaryl group evenly or randomly distributed along the polymer,
L2 is an aryl or a heteroaryl group located on the ends of the polymer,
FL is a fluorescent moiety,
G1 and G2 stand for hydrogen, halogen or an antigen recognizing moiety, with the provision that at least one of G1 or G2 is an antigen recognizing moiety,
a is 10 to 100 mol %,
b is 0.1 to 50 mol %
c is 0 to 90 mol %
d is 1 to 10,000; with the provision that a+b+c=100 mol %
characterized in that AR is connected in the polymer chain via the 2,2′ or 3,3′ or 5,5′ or 6,6′ or 7,7′ or 8,8′position according to general formula (II) 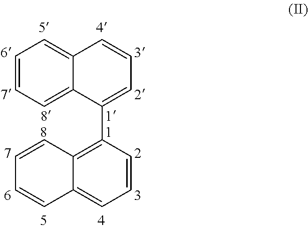
wherein position 2,2′; 3,3′; 4,4′; 5,5′; 6,6′; 7,7′; 8,8′ are same or different substituted with residues selected from the group consisting of H, SO₂CF₃, SO₂Rₐ, CF₃, CCl₃, CN, SO₃H, NO₂, NRaRbRc+, CHO, CORa, CO₂Ra, COCl, CONRaRb, F, Cl, Br, I, Ra, ORa, SRa, OCORa, NRaRb, NHCORa, CCRa, aryl-, heteroaryl-, C₆H₄ORa or C₆H₄NRaRb, with Rₐ-c independently hydrogen, alkyl-, alkenyl-, alkinyl-, heteroalkyl-, aryl-, heteroaryl-, cycloalkyl-, alkylcycloalkyl-, heteroalkylcycloalkyl-, heterocycloalkyl-, aralkyl- or a heteroaralkyl residue or (CH2)x,(OCH2CH2)yO(CH2)zCH3 where
x is an integer from 0-20 and
y is an integer from 0 to 50;
z is an integer from 0-20,
or two residues both as part of a cycloalkyl- or heterocycloalkyl ring system.

2. Conjugate according to claim 1 characterized in that the conjugate has a main chirality with AR provided mainly as R- or S-stereo isomer.

3. Conjugate according to claim 1 characterized in that MU is a polymer modifying unit or band gap modifying unit that is evenly or randomly distributed along the polymer main chain and is optionally substituted with one or more optionally substituted substituents selected from halogen, hydroxyl, C1-C12 alkyl, C2-C12 alkene, C2-C12 alkyne, C3-C12 cycloalkyl, C1-C12 haloalkyl, C1-C12 alkoxy, C2-C18 (hetero)aryloxy, C2-C18 (hetero) arylamino, a C2-C18 (hetero)aryl group and (CH2)x,(OCH2CH2)yO(CH2)zCH3 where
x is an integer from 0-20 and
y is an integer from 0 to 50;
z is an integer from 0-20.

4. The Conjugate according to claim 1, characterized in that L1 is an aryl or a heteroaryl group evenly or randomly distributed along the polymer main chain and is substituted with one or more pendant chains terminated with: i) a functional group selected from amine, carbamate, carboxylic acid, carboxylate, maleimide, activated ester, N-hydroxysuccinimidyl, hydrazine, hydrazide, azide, alkyne, aldehyde, thiol, and protected groups thereof for conjugation to a molecule or biomolecule; or ii) an attached conjugated organic dye as acceptor dye, or iii) a biomolecule.

5. The conjugate according to claim 1, characterized in that L2 is an aryl or a heteroaryl group located on the ends of the polymer main chain and is substituted with one or more pendant chains terminated with: i) a functional group selected from amine, carbamate, carboxylic acid, carboxylate, maleimide, activated ester, N-hydroxysuccinimidyl, hydrazine, hydrazide, azide, alkyne, aldehyde, thiol, and protected groups thereof for conjugation to a molecule or biomolecule; or ii) an attached organic dye as acceptor dye, or iii) a biomolecule.

6. The conjugate according to claim 1, characterized in that L2 is selected from one or more of the following structures:

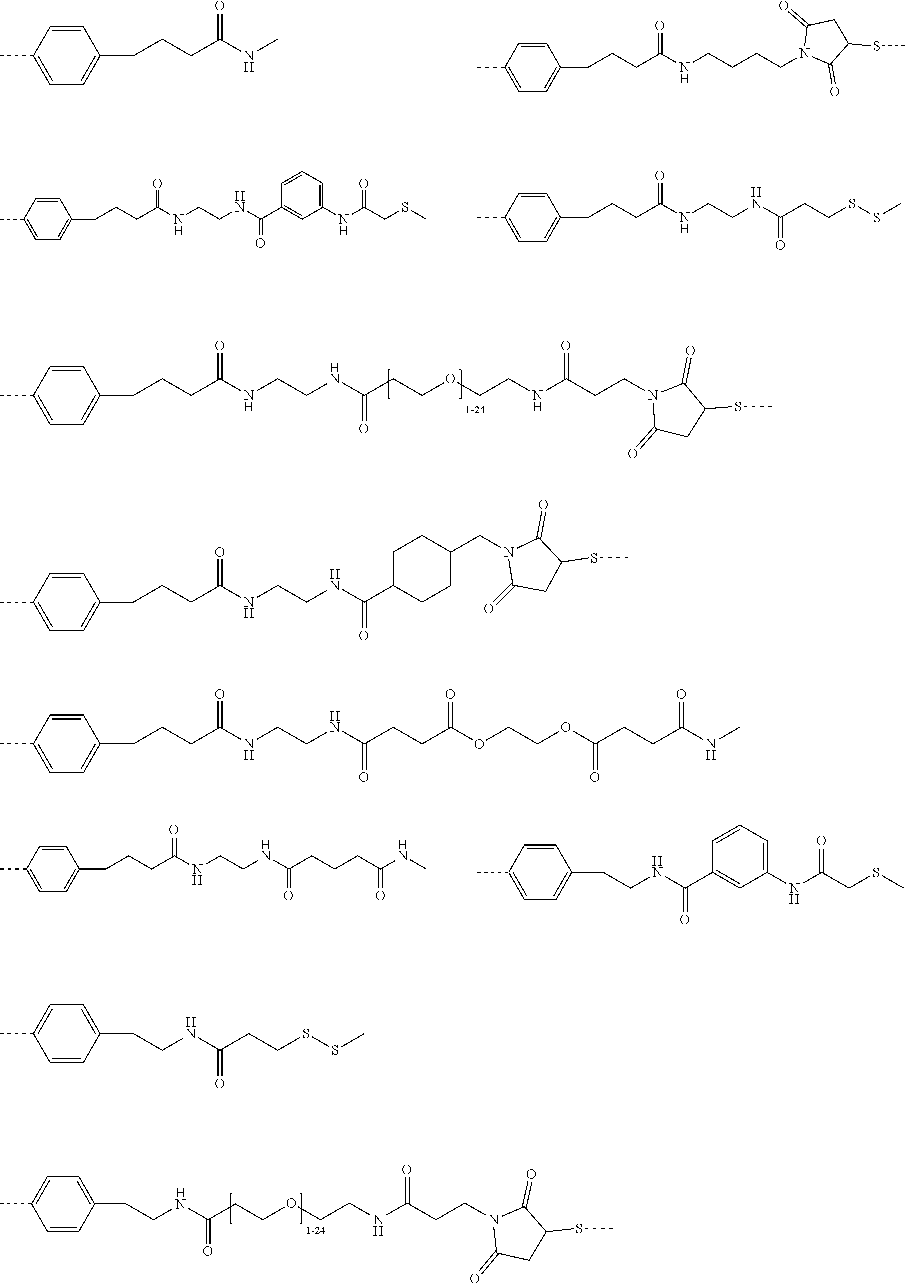

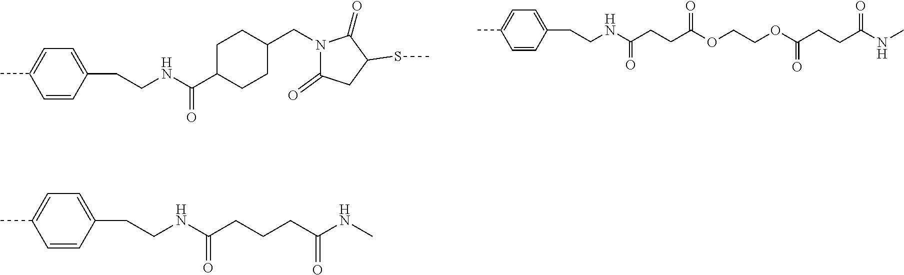

7. The Conjugate according to claim 1, characterized in that FL is selected from the group consisting of Fluorescein, Fluorescein-Derivatives, Rhodamine, Tetramethylrhodamine, Silicon-Rhodamine (SiR), Coumarines, Resorufines, Pyrenes, Anthracenes, Phenylenes, Phthalocyanines, Cyanines, Xanthenes, Amidopyrylium-Fluorophores, Oxazine, Quadrain-Farbstoffe, Carbopyronine, 7-Nitrobenz-2-Oxa-1,3-Diazol (NBD) Fluorophore, BODIPY™ Fluorophores (Molecular Probes, Inc.), ALEXA™ Fluorophore (Molecular Probes, Inc.), DY™ Fluorophores (Dyomics GmbH), Benzopyrylium Fluorophores, Benzopyrylium-Polymethine Fluorophores, Lanthanid-Chelate, Metalloporhyrines, Rhodol dyes, Carborhodol dyes, Naphthalimides and Porphyrines.

8. The Conjugate according to claim 1, characterized in that at least two positions 2,2′; 3,3′; 4,4′; 5,5′; 6,6′ 7,7′ and 8,8′ are substituted with residues according to general formula (III)

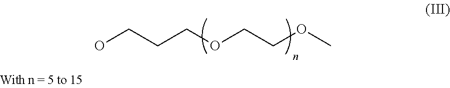

9. The Conjugate according to claim 1, characterized in that G1 and G2 are both independently chosen from the group consisting of hydrogen, halogen or an antigen recognizing moiety, with at least one antigen recognizing, selected from the group consisting of: An antibody, an fragmented antibody, an fragmented antibody derivative, peptide/MHC-complexes, receptors for cell adhesion or costimulatory molecules, receptor ligands, antigens, hapten binders, avidin, streptavidin, travidin, aptamers, primers and ligase substrates, peptide/MHC complex targeting TCR molecules, cell adhesion receptor molecules, receptors for costimulatory molecules or artificial engineered binding molecules.

10. A method for detecting a target moiety in a sample of biological specimens characterized by the steps
a) contacting the sample of biological specimens with at least one conjugate (I), thereby labeling the target moiety recognized by an antigen recognizing moiety with the conjugate (I);
b) exciting the labelled target moieties with light having a wavelength within the absorbance spectrum of the multichromophore CP-FL or the fluorescent moiety FL;
c) detecting the labelled target moieties by detecting the fluorescence radiation emitted by the fluorescent moiety FL

11. The method according to claim 10 characterized in that after step c), the fluorescent moiety FL of the labelled target moieties is degraded by irradiating the conjugate with light having a wavelength within the absorbance spectrum of fluorescent moiety FL for a time sufficient to deliver enough energy to reduce the fluorescence radiation emitted by the fluorescent moiety FL at least by 75% of the initial fluorescence radiation.

12. Use of the conjugate according to claim 1 in fluorescence microscopy, flow cytometer, fluorescence spectroscopy, cell separation, pathology or histology.
